SEQUENCE LISTING (153 sequences)

SEQ ID NO: 1 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 2 - protein, 95 aa, Homo sapiens; identical to SEQ ID NO: 1

SEQ ID NO: 3 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 4 - protein, 95 aa, Homo sapiens; identical to SEQ ID NO: 3

SEQ ID NO: 5 - protein, 95 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAP

SEQ ID NO: 6 - protein, 95 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYP

SEQ ID NO: 7 - protein, 95 aa, Homo sapiens
NIQMTQSPSAMSASVGDRVTITCRARQGISNYLAWFQQKPGKVPKHLIYAASSLQSGVPSRFSGSGSGTEFTLTISSLQPEDFATYYCLQHNSYP

SEQ ID NO: 8 - protein, 95 aa, Homo sapiens
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

SEQ ID NO: 9 - protein, 95 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 10 - protein, 95 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 11 - protein, 95 aa, Homo sapiens; identical to SEQ ID NO: 10

SEQ ID NO: 12 - protein, 95 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 13 - protein, 95 aa, Homo sapiens; identical to SEQ ID NO: 12

SEQ ID NO: 14 - protein, 95 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYP

SEQ ID NO: 15 - protein, 95 aa, Homo sapiens
AIRMTQSPFSLSASVGDRVTITCWASQGISSYLAWYQQKPAKAPKLFIYYASSLQSGVPSRFSGSGSGTDFTLTISSLQPEDFATYYCQQYYSTP

SEQ ID NO: 16 - protein, 95 aa, Homo sapiens
AIRMTQSPSSFSASTGDRVTITCRASQGISSYLAWYQQKPGKAPKLLIYAASTLQSGVPSRFSGSGSGTDFTLTISCLQSEDFATYYCQQYYSYP

SEQ ID NO: 17 - protein, 95 aa, Homo sapiens
VIMMTQSPSLLSASTGDRVTISCRMSQGISSYLAWYQQKPGKAPELLIYAASTLQSGVPSRFSGSGSGTDFTLTISCLQSEDFATYYCQQYYSFP

SEQ ID NO: 18 - protein, 95 aa, Homo sapiens
AIQMTQSPSSLSASVGDRVTITCRASQGIRNDLGWYQQKPGKAPKLLIYAASSLQSGVPSRFSGSGSGTDFTLTISSLQPEDFATYYCLQDYNYP

SEQ ID NO: 19 - protein, 95 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYCQQYNSYS

SEQ ID NO: 20 - protein, 101 aa, Homo sapiens
DIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFP

SEQ ID NO: 21 - protein, 101 aa, Homo sapiens; identical to SEQ ID NO: 20

SEQ ID NO: 22 - protein, 100 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWP

SEQ ID NO: 23 - protein, 100 aa, Homo sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCMQGTHWP

SEQ ID NO: 24 - protein, 100 aa, Homo sapiens
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYCMQGIHLP

SEQ ID NO: 25 - protein, 100 aa, Homo sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLP

SEQ ID NO: 26 - protein, 100 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTP

SEQ ID NO: 27 - protein, 100 aa, Homo sapiens; identical to SEQ ID NO: 26

SEQ ID NO: 28 - protein, 100 aa, Homo sapiens
DIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFP

SEQ ID NO: 29 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 30 - protein, 96 aa, Homo sapiens
EIVLTQSPATLSLSPGERATLSCGASQSVSSSYLAWYQQKPGLAPRLLIYDASSRATGIPDRFSGSGSGTDFTLTISRLEPEDFAVYYCQQYGSSP

SEQ ID NO: 31 - protein, 95 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 32 - protein, 95 aa, Homo sapiens; identical to SEQ ID NO: 31

SEQ ID NO: 33 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 34 - protein, 95 aa, Homo sapiens
EIVLTQSPATLSLSPGERATLSCRASQGVSSYLAWYQQKPGQAPRLLIYDASNRATGIPARFSGSGPGTDFTLTISSLEPEDFAVYYCQQRSNWH

SEQ ID NO: 35 - protein, 96 aa, Homo sapiens
EIVMTQSPATLSLSPGERATLSCRASQSVSSSYLSWYQQKPGQAPRLLIYGASTRATGIPARFSGSGSGTDFTLTISSLQPEDFAVYYCQQDYNLP

SEQ ID NO: 36 - protein, 101 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 37 - protein, 95 aa, Homo sapiens
ETTLTQSPAFMSATPGDKVNISCKASQDIDDDMNWYQQKPGEAAIFIIQEATTLVPGIPPRFSGSGYGTDFTLTINNIESEDAAYYFCLQHDNFP

SEQ ID NO: 38 - protein, 95 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 39 - protein, 95 aa, Homo sapiens; identical to SEQ ID NO: 38

SEQ ID NO: 40 - protein, 95 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHP

SEQ ID NO: 41 - protein, 98 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 42 - protein, 98 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 43 - protein, 98 aa, Homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 44 - protein, 98 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 45 - protein, 98 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 46 - protein, 98 aa, Homo sapiens
QMQLVQSGAEVKKTGSSVKVSCKASGYTFTYRYLHWVRQAPGQALEWMGWITPFNGNTNYAQKFQDRVTITRDRSMSTAYMELSSLRSEDTAMYYCAR

SEQ ID NO: 47 - protein, 98 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 48 - protein, 98 aa, Homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 49 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 50 - protein, 98 aa, Homo sapiens
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAR

SEQ ID NO: 51 - protein, 98 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 52 - protein, 100 aa, Homo sapiens
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHR

SEQ ID NO: 53 - protein, 100 aa, Homo sapiens
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARI

SEQ ID NO: 54 - protein, 100 aa, Homo sapiens
QVTLKESGPALVKPTQTLTLTCTFSGFSLSTSGMRVSWIRQPPGKALEWLARIDWDDDKFYSTSLKTRLTISKDTSKNQVVLTMTNMDPVDTATYYCARI

SEQ ID NO: 55 - protein, 98 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 56 - protein, 99 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKD

SEQ ID NO: 57 - protein, 98 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 58 - protein, 97 aa, Homo sapiens
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCAR

SEQ ID NO: 59 - protein, 100 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 60 - protein, 98 aa, Homo sapiens
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCAR

SEQ ID NO: 61 - protein, 98 aa, Homo sapiens
EVQLVESGGGLVKPGGSLRLSCAASGFTFSSYSMNWVRQAPGKGLEWVSSISSSSSYTYYADSVKGRFTISRDNAKNSLYLQMNSLRAEDTAVYYCAR

SEQ ID NO: 62 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 63 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 64 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 65 - protein, 98 aa, Homo sapiens; identical to SEQ ID NO: 63

SEQ ID NO: 66 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 67 - protein, 99 aa, Homo sapiens
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAKD

SEQ ID NO: 68 - protein, 98 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCAR

SEQ ID NO: 69 - protein, 100 aa, Homo sapiens
EVQLVESGGGLVQPGRSLRLSCTASGFTFGDYAMSWFRQAPGKGLEWVGFIRSKAYGGTTEYTASVKGRFTISRDGSKSIAYLQMNSLKTEDTAVYYCTR

SEQ ID NO: 70 - protein, 97 aa, Homo sapiens
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 71 - protein, 98 aa, Homo sapiens
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCAR

SEQ ID NO: 72 - protein, 97 aa, Homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 73 - protein, 100 aa, Homo sapiens
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAR

SEQ ID NO: 74 - protein, 100 aa, Homo sapiens
EVQLVESGGGLVQPGGSLKLSCAASGFTFSGSAMHWVRQAPGKGLEWVGRIRSKANSYATAYAASVKGRFTISRDDSKNTAYLQMNSLKTEDTAVYYCTR

SEQ ID NO: 75 - protein, 98 aa, Homo sapiens
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAR

SEQ ID NO: 76 - protein, 96 aa, Homo sapiens
EVQLVESRGVLVQPGGSLRLSCAASGFTVSSNEMSWVRQAPGKGLEWVSSISGGSTYYADSRKGRFTISRDNSKNTLHLQMNSLRAEDTAVYYCKK

SEQ ID NO: 77 - protein, 98 aa, Homo sapiens
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCAR

SEQ ID NO: 78 - protein, 98 aa, Homo sapiens
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCAR

SEQ ID NO: 79 - protein, 99 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCAR

SEQ ID NO: 80 - protein, 99 aa, Homo sapiens
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCAR

SEQ ID NO: 81 - protein, 99 aa, Homo sapiens
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAR

SEQ ID NO: 82 - protein, 99 aa, Homo sapiens; identical to SEQ ID NO: 79

SEQ ID NO: 83 - protein, 97 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 84 - protein, 99 aa, Homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 85 - protein, 97 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 86 - protein, 99 aa, Homo sapiens
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAR

SEQ ID NO: 87 - protein, 98 aa, Homo sapiens
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAR

SEQ ID NO: 88 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 89 - protein, 98 aa, Homo sapiens
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCAR

SEQ ID NO: 90 - protein, 101 aa, Homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAR

SEQ ID NO: 91 - protein, 98 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQICSLKAEDTAVYYCAR

SEQ ID NO: 92 - protein, 98 aa, Homo sapiens
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNG

SEQ ID NO: 93 - protein, 99 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 94 - protein, 98 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNG

SEQ ID NO: 95 - protein, 98 aa, Homo sapiens
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSG

SEQ ID NO: 96 - protein, 98 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSA

SEQ ID NO: 97 - protein, 99 aa, Homo sapiens
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNF

SEQ ID NO: 98 - protein, 99 aa, Homo sapiens
QSALTQPRSVSGSPGQSVTISCTGTSSDVGGYNYVSWYQQHPGKAPKLMIYDVSKRPSGVPDRFSGSKSGNTASLTISGLQAEDEADYYCCSYAGSYTF

SEQ ID NO: 99 - protein, 99 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTL

SEQ ID NO: 100 - protein, 99 aa, Homo sapiens
QSALTQPPSVSGSPGQSVTISCTGTSSDVGSYNRVSWYQQPPGTAPKLMIYEVSNRPSGVPDRFSGSKSGNTASLTISGLQAEDEADYYCSLYTSSSTF

SEQ ID NO: 101 - protein, 99 aa, Homo sapiens
QSALTQPASVSGSPGQSITISCTGTSSDVGSYNLVSWYQQHPGKAPKLMIYEVSKRPSGVSNRFSGSKSGNTASLTISGLQAEDEADYYCCSYAGSSTF

SEQ ID NO: 102 - protein, 95 aa, Homo sapiens
SYELTQPPSVSVSPGQTASITCSGDKLGDKYACWYQQKPGQSPVLVIYQDSKRPSGIPERFSGSNSGNTATLTISGTQAMDEADYYCQAWDSSTA

SEQ ID NO: 103 - protein, 95 aa, Homo sapiens
SYELTQPLSVSVALGQTARITCGGNNIGSKNVHWYQQKPGQAPVLVIYRDSNRPSGIPERFSGSNSGNTATLTISRAQAGDEADYYCQVWDSSTA

SEQ ID NO: 104 - protein, 96 aa, Homo sapiens
SYELTQPPSVSVSPGQTARITCSGDALPKKYAYWYQQKSGQAPVLVIYEDSKRPSGIPERFSGSSSGTMATLTISGAQVEDEADYYCYSTDSSGNH

SEQ ID NO: 105 - protein, 96 aa, Homo sapiens
SYELTQPPSVSVSLGQMARITCSGEALPKKYAYWYQQKPGQFPVLVIYKDSERPSGIPERFSGSSSGTIVTLTISGVQAEDEADYYCLSADSSGTY

SEQ ID NO: 106 - protein, 96 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNH

SEQ ID NO: 107 - protein, 96 aa, Homo sapiens
SYVLTQPPSVSVAPGKTARITCGGNNIGSKSVHWYQQKPGQAPVLVIYYDSDRPSGIPERFSGSNSGNTATLTISRVEAGDEADYYCQVWDSSSDH

SEQ ID NO: 108 - protein, 94 aa, Homo sapiens
SYELTQLPSVSVSPGQTARITCSGDVLGENYADWYQQKPGQAPELVIYEDSERYPGIPERFSGSTSGNTTTLTISRVLTEDEADYYCLSGDEDN

SEQ ID NO: 109 - protein, 96 aa, Homo sapiens
SYELMQPPSVSVSPGQTARITCSGDALPKQYAYWYQQKPGQAPVLVIYKDSERPSGIPERFSGSSSGTTVTLTISGVQAEDEADYYCQSADSSGTY

SEQ ID NO: 110 - protein, 94 aa, Homo sapiens
SYELTQPSSVSVSPGQTARITCSGDVLAKKYARWFQQKPGQAPVLVIYKDSERPSGIPERFSGSSSGTTVTLTISGAQVEDEADYYCYSAADNN

SEQ ID NO: 111 - protein, 103 aa, Homo sapiens
LPVLTQPPSASALLGASIKLTCTLSSEHSTYTIEWYQQRPGRSPQYIMKVKSDGSHSKGDGIPDRFMGSSSGADRYLTFSNLQSDDEAEYHCGESHTIDGQVG

SEQ ID NO: 112 - protein, 99 aa, Homo sapiens
QPVLTQSSSASASLGSSVKLTCTLSSGHSSYIIAWHQQQPGKAPRYLMKLEGSGSYNKGSGVPDRFSGSSSGADRYLTISNLQLEDEADYYCETWDSNT

SEQ ID NO: 113 - protein, 99 aa, Homo sapiens
QLVLTQSPSASASLGASVKLTCTLSSGHSSYAIAWHQQQPEKGPRYLMKLNSDGSHSKGDGIPDRFSGSSSGAERYLTISSLQSEDEADYYCQTWGTGI

SEQ ID NO: 114 - protein, 104 aa, Homo sapiens
QPVLTQPPSSSASPGESARLTCTLPSDINVGSYNIYWYQQKPGSPPRYLLYYYSDSDKGQGSGVPSRFSGSKDASANTGILLISGLQSEDEADYYCMIWPSNAS

SEQ ID NO: 115 - protein, 104 aa, Homo sapiens
QAVLTQPASLSASPGASASLTCTLRSGINVGTYRIYWYQQKPGSPPQYLLRYKSDSDKQQGSGVPSRFSGSKDASANAGILLISGLQSEDEADYYCMIWHSSAS

SEQ ID NO: 116 - protein, 105 aa, Homo sapiens
QPVLTQPSSHSASSGASVRLTCMLSSGFSVGDFWIRWYQQKPGNPPRYLLYYHSDSNKGQGSGVPSRFSGSNDASANAGILRISGLQPEDEADYYCGTWHSNSKT

SEQ ID NO: 117 - protein, 98 aa, Homo sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSN

SEQ ID NO: 118 - protein, 98 aa, Homo sapiens
QTVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAQ

SEQ ID NO: 119 - protein, 98 aa, Homo sapiens
QAVVTQEPSLTVSPGGTVTLTCGSSTGAVTSGHYPYWFQQKPGQAPRTLIYDTSNKHSWTPARFSGSLLGGKAALTLSGAQPEDEAEYYCLLSYSGAR

SEQ ID NO: 120 - protein, 98 aa, Homo sapiens
QTVVTQEPSFSVSPGGTVTLTCGLSSGSVSTSYYPSWYQQTPGQAPRTLIYSTNTRSSGVPDRFSGSILGNKAALTITGAQADDESDYYCVLYMGSGI

SEQ ID NO: 121 - protein, 104 aa, Homo sapiens
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFV

SEQ ID NO: 122 - protein, 98 aa, Homo sapiens
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLSA

SEQ ID NO: 123-138 - skipped (16 entries)

SEQ ID NO: 139 - protein, 17 aa, Homo sapiens
AEYFQHWGQGTLVTVSS

SEQ ID NO: 140 - protein, 17 aa, Homo sapiens
YWYFDLWGRGTLVTVSS

SEQ ID NO: 141 - protein, 15 aa, Homo sapiens
AFDIWGQGTMVTVSS

SEQ ID NO: 142 - protein, 15 aa, Homo sapiens
YFDYWGQGTLVTVSS

SEQ ID NO: 143 - protein, 16 aa, Homo sapiens
NWFDPWGQGTLVTVSS

SEQ ID NO: 144 - protein, 20 aa, Homo sapiens
YYYYYGMDVWGQGTTVTVSS

SEQ ID NO: 145 - protein, 12 aa, Homo sapiens
WTFGQGTKVEIK

SEQ ID NO: 146 - protein, 12 aa, Homo sapiens
YTFGQGTKLEIK

SEQ ID NO: 147 - protein, 12 aa, Homo sapiens
FTFGPGTKVDIK

SEQ ID NO: 148 - protein, 12 aa, Homo sapiens
LTFGGGTKVEIK

SEQ ID NO: 149 - protein, 12 aa, Homo sapiens
ITFGQGTRLEIK

SEQ ID NO: 150 - protein, 12 aa, Homo sapiens
YVFGTGTKVTVL

SEQ ID NO: 151 - protein, 12 aa, Homo sapiens
VVFGGGTKLTVL

SEQ ID NO: 152 - protein, 12 aa, Homo sapiens
VVFGGGTKLTVL

SEQ ID NO: 153 - protein, 12 aa, Homo sapiens
AVFGGGTQLTVL

(19) United States
(12) Patent Application Publication
(10) Pub. No.: US 2011/0313134 A1
(43) Pub. Date: Dec. 22, 2011

(54) ENGINEERED ANTIBODIES WITH REDUCED IMMUNOGENICITY AND METHODS OF MAKING

(75) Inventor: Russell P. Rother, Oklahoma City, OK (US)

(73) Assignee: ALEXION PHARMACEUTICALS, INC., Cheshire, CT (US)

(21) Appl. No.: 13/128,127

(22) PCT Filed: Nov. 6, 2009
(86) PCT No.: PCT/US2009/063574

§ 371 Date: Aug. 24, 2011

Related U.S. Application Data
(60) Provisional application No. 61/198,466, filed on Nov. 6, 2008.

(51) Int. Cl. C07K 16/46

(52) U.S. Cl. 530/387.3

(57) ABSTRACT

Hybrid antibodies and antibody binding fragments thereof having decreased immunogenicity and methods of making them are provided. The methods involve replacing one or more amino acid residues within at least one donor framework region of a hybrid antibody or antigen binding fragment thereof that has undergone somatic hypermutation with the amino acid residue from the corresponding position of a germline framework sequence. Also provided are hybrid antibodies or antigen binding fragments thereof containing at least two donor framework regions that are derived from the same germline gene family or germline gene family member and wherein at least one amino acid residue within a framework region has been replaced with an amino acid residue from the corresponding position within a germline framework region. The hybrid antibodies or antigen binding fragments thereof may contain human framework regions and nonhuman CDRs.

### RELATED APPLICATIONS

This application claims the benefit of U.S. Provisional Application No. 61/198,466, filed Nov. 6, 2008, which application is hereby incorporated by reference in its entirety.

### BACKGROUND

Antibodies are proteins produced by lymphocytes known as B cells in vertebrates in response to stimulation by antigens. The basic structural unit of an antibody (a.k.a. immunoglobulin (Ig)) molecule consists of four polypeptide chains which come together in the shape of a capital letter “Y”. Two of the four chains are identical light (L) chains and two are identical heavy (H) chains. There are five different kinds (isotypes) of heavy chains which divide antibodies into five classes, namely, IgA, IgD, IgE, IgG and IgM. In addition, there are two different isotypes of light chains designated κ and λ. Each class of heavy chains can combine with either of the light chains. The heavy and light chains each contain a variable region (VH and VL, respectively) that is involved in antigen binding and a constant (C) region. The antigen binding site is composed of six hypervariable regions (a.k.a. complementarity determining regions (CDRs)). Three CDRs from the heavy chain and three CDRs from the light chain are respectively positioned between four relatively conserved anti-parallel β-sheets which are called framework regions (FR1, FR2, FR3 and FR4), on each chain. By convention, numbering systems have been utilized to designate the location of the component parts of VH and VL chains. The Kabat definition is based on sequence variability and the Chothia definition is based on the location of structural loop regions.

For each type of Ig chain synthesized by B cells, there is a separate pool of gene segments, known as germline genes, from which a single polypeptide chain is synthesized. Each pool is located on a different chromosome and typically contains a relatively large number of gene segments encoding the V region and a smaller number of gene segments encoding the C region. Each light chain V region is encoded by a nucleic acid sequence assembled from two kinds of germline gene segments, i.e., a long V gene segment, a short joining (J) gene segment, and a C segment. The heavy chain is encoded by four kinds of germline gene segments, three for the variable region and one for the constant region. The three germline gene segments that encode the heavy chain variable region are a V segment, a J segment and a diversity (D) segment. Human germline V, D and J gene sequences have been characterized. There are approximately fifty-one human germline VH gene segments (such “segments” are also referred to herein as germline gene family members) that are classified into seven germline gene families (VH1-VH7) based on sequence homology of at least 80%. See, e.g., Matsuda, et al. J. Exp. Med. (1998) 188:2151-2162. Through somatic hypermutation (or antibody maturation), each germline gene family member can give rise to multiple species of immunoglobulins that are derivatives of a given germline gene family member. The germline gene family member from which a somatically mutated antibody is derived may be determined by aligning the sequence of the somatically mutated antibody with the sequences of germline genes to evaluate the sequence identity with the germline gene family members.

The first two CDRs and three framework regions of the heavy chain variable region are encoded by VH. CDR3 is encoded by a few nucleotides of VH, all of DH and part of JH, while FR4 is encoded by the remainder of the JH gene segment. Similarly, with regard to light chains, V Kappa (Vκ) or V lambda (Vλ) gene segments (e.g., germline gene family members) encode the first two CDR and three framework regions of the V region along with a few residues of CDR3. J Kappa (Jκ) and J Lambda (Jλ) segments encode the remainder of the CDR3 region in a Vκ or Vλ region, respectively. DNA encoding the κ chain includes approximately forty Vκ segments (germline gene family members) that are classified into six families (VκI-VκVI) based on sequence homology. DNA encoding the λ chain includes approximately thirty-one V λ segments (germline gene family members) that are classified into ten families. See FIGS. 1, 2, 3 and 4.

Antibodies and antibody fragments have become promising therapeutic agents in connection with various human diseases in both acute and chronic settings. There are several methods being utilized to generate antibodies including hybridoma technology, bacterial display, ribosome display, yeast display, and recombinant expression of human antibody fragments on the surface of replicative bacteriophage. Monoclonal antibodies (mAbs), which may be produced by hybridomas, have been applied successfully as diagnostics for many years, but their use as therapeutic agents is just emerging. The vast majority of mAbs are of non-human (largely rodent) origin, posing the problem of immunogenicity in humans. When antibodies of rodent origin are administered to humans, anti-rodent antibodies are generated which result in enhanced clearance of the rodent antibody from the serum, blocking of its therapeutic effect and hypersensitivity reactions. These limitations have prompted the development of engineering technologies known as “humanization”.

The first humanization strategies were based on the knowledge that heavy and light chain variable domains are responsible for binding to antigen, and the constant domains for effector function. Chimeric antibodies were created, for example, by transplanting the variable domains of a rodent mAb to the constant domains of human antibodies (e.g. Neuberger M S, et al., Nature 314, 268-70, 1985 and Takeda, et al., Nature 314, 452-4, 1985). Although these chimeric antibodies induce better effector functions in humans and exhibit reduced immunogenicity, the rodent variable region still poses a risk of inducing an immune response. When it was recognized that the variable domains consist of a beta sheet framework surmounted by antigen-binding loops (complementarity determining regions or CDR's), humanized antibodies were designed to contain the rodent CDR's grafted onto a human framework. Several different antigen-binding sites were successfully transferred to a single human framework, often using an antibody where the entire human framework regions have the closest homology to the rodent sequence (e.g., Jones P T, et al., Nature 321, 522-5, 1986; Riechmann L. et al., Nature 332, 323-327, 1988; and Sato K. et al., Mol. Immunol. 31, 371-8, 1994). Alternatively, consensus human frameworks were built based on several human heavy chains (e.g., Carter P. et al., Proc. Nat. Acad. Sci. USA 89, 487-99, 1992). However, simple CDR grafting often resulted in loss of antigen affinity. Other possible interactions between the beta-sheet framework and the loops had to be considered to recreate the antigen binding site (Chothia C, et al., Mol. Biol. 196, 901-917, 1987).

Comparison of the essential framework residues required in humanization of several antibodies, as well as computer modeling based on antibody crystal structures revealed a set of framework residues termed as “Vernier zone residues” (Foote J., et al., Mol Biol 224, 487-99, 1992) that most likely contributes to the integrity of the binding site. In addition, several residues in the VH-VL interface zone might be important in maintaining affinity for the antigen (Santos A D, et al., Prog. Nucleic Acid Res Mol Biol 60, 169-94 1998). Initially, framework residues were stepwise mutated back to the rodent sequence (Kettleborough C A, et al. Protein Engin. 4, 773-783, 1991). However, this mutation approach is very time-consuming and cannot cover every important residue.

For any particular antibody a small set of changes may suffice to optimize binding, yet it is difficult to select from the set of Vernier and VH/VL residues. Combinatorial library approaches combined with selection technologies (such as phage display) revolutionized humanization technologies by creating a library of humanized molecules that represents alternatives between rodent and human sequence in all important framework residues and allows for simultaneous determination of binding activity of all humanized forms (e.g. Rosok M J, J Biol Chem, 271, 22611-8, 1996 and Baca M, et al. J Biol Chem 272, 10678-84, 1997).

Clearly, there exists a need for improved methods of producing humanized antibodies with reduced immunogenicity while maintaining an optimum binding profile that can be administered to a target species for therapeutic and diagnostic purposes.

### SUMMARY

In one aspect, a method for producing a hybrid antibody variable domain or fragment thereof is provided, wherein the method comprises (i) selecting a framework region from the group consisting of FR1, FR2, and FR3 within a variable region of an initial antibody having specificity for a target; (ii) comparing the selected framework region against candidate donor framework sequences from said target species to identify a first donor framework sequence having a high degree of homology with the selected framework region; (iii) comparing the first donor framework sequence to germline sequences of said target species to identify a first germline framework sequence having a high degree of homology with the first donor framework sequence; (iv) identifying at least one amino acid residue within the first donor framework sequence that is different from the amino acid residue at the corresponding position in the first germline framework sequence; and (v) constructing a hybrid antibody variable domain or fragment thereof comprising the complementarity determining regions (CDRs) of the initial antibody and the first donor framework sequence, wherein the at least one amino acid residue within the first donor framework sequence is replaced with the amino acid residue at the corresponding position in the first germline framework sequence.

In certain embodiments, the method further comprises selecting a second framework region from the group consisting of FR1, FR2, and FR3 of the initial antibody, comparing the selected framework region against candidate donor framework sequences from said target species to identify a second donor framework sequence having a high degree of homology with the selected framework region, and constructing a hybrid antibody variable domain or fragment thereof comprising the CDRs of the initial antibody and the donor framework sequences.

In certain embodiments, the methods may additionally comprise, in no particular order, comparing the second donor framework sequence to germline sequences of said target species to identify a second germline framework sequence having a high degree of homology with the second framework sequence, and replacing at least one amino acid residue within the second donor framework sequence with the amino acid residue at the corresponding position in the second germline framework sequence.

In certain embodiments, said first and second donor sequences are from two different antibodies that belong to the same germline gene family.

In certain embodiments, the method further comprises selecting a third framework region from the group consisting of FR1, FR2, and FR3 of the initial antibody, comparing the selected framework region against candidate donor framework sequences from said target species to identify a third donor framework sequence having a high degree of homology with the selected framework region; and constructing a hybrid antibody variable domain or fragment thereof comprising the CDRs of the initial antibody and the donor framework sequences.

In certain embodiment, the methods may additionally comprise, in no particular order, comparing the third donor framework sequence to germline sequences of said target species to identify a third germline framework sequence having a high degree of homology with the third framework sequence, and replacing at least one amino acid residue within the third donor framework sequence with the amino acid residue at the corresponding position in the third germline framework sequence.

In certain embodiments, said third donor sequence belongs to the same germline gene family as the first and second germline gene family.

In certain embodiments, the method further comprises selecting a fourth framework region which is FR4 of the initial antibody, comparing the selected framework region against candidate donor framework sequences from said target species to identify a fourth donor framework sequence having a high degree of homology with the selected framework region, and constructing a hybrid antibody variable domain or fragment thereof comprising the CDRs of the initial antibody and the donor framework sequences.

In certain embodiments, the methods may additionally comprise, in no particular order, comparing the fourth donor framework sequence to germline sequences of said target species to identify a fourth germline framework sequence having a high degree of homology with the fourth framework sequence, and replacing at least one amino acid residue within the fourth donor framework sequence with the amino acid residue at the corresponding position in the fourth germline framework sequence.

In another aspect, a method for producing a hybrid antibody variable domain or fragment thereof is provided, wherein the method comprises (i) selecting a framework region from the group consisting of FR1, FR2, and FR3 within a variable region of an initial antibody having specificity for a target; (ii) comparing the selected framework region of the initial antibody against candidate donor variable region sequences from said target species to identify a first donor framework sequence having a high degree of homology with the selected framework region; (iii) constructing a hybrid antibody variable domain or fragment thereof comprising the complementarity determining regions (CDRs) of the initial antibody and the first donor framework sequence; (iv) comparing the first donor framework sequence to germline variable region sequences of said target species to identify a first germline framework sequence having a high degree of homology with the first donor framework sequence; and (v) selectively substituting at least one amino acid residue within the first donor framework sequence with the amino acid residue at the corresponding position in the first germline framework sequence.

In certain embodiments, the method further comprises selecting a second framework region from the group consisting of FR1, FR2, and FR3 of the initial antibody, comparing the selected framework region against candidate donor framework sequences from said target species to identify a second donor framework sequence having a high degree of homology with the selected framework region; and constructing a hybrid antibody variable domain or fragment thereof comprising the CDRs of the initial antibody and the donor framework sequences.

In certain embodiments, the methods may additionally comprise, in no particular order, comparing the second donor framework sequence to germline sequences of said target species to identify a second germline framework sequence having a high degree of homology with the second framework sequence, and replacing at least one amino acid residue within the second donor framework sequence with the amino acid residue at the corresponding position in the second germline framework sequence.

In certain embodiments, said first and second donor sequences are from two different antibodies that belong to the same germline gene family.

In certain embodiments, the method further comprises selecting a third framework region from the group consisting of FR1, FR2, and FR3 of the initial antibody, comparing the selected framework region against candidate donor framework sequences from said target species to identify a third donor framework sequence having a high degree of homology with the selected framework region; and constructing a hybrid antibody variable domain or fragment thereof comprising the CDRs of the initial antibody and the donor framework sequences.

In certain embodiments, the methods may additionally comprise, in no particular order, comparing the third donor framework sequence to germline sequences of said target species to identify a third germline framework sequence having a high degree of homology with the third framework sequence, and replacing at least one amino acid residue within the third donor framework sequence with the amino acid residue at the corresponding position in the third germline framework sequence.

In certain embodiments, said third donor sequence belong to the same germline gene family as the first and second donor sequences.

In certain embodiments, the method further comprises selecting a fourth framework region which is FR4 of the initial antibody, comparing the selected framework region against candidate donor framework sequences from said target species to identify a fourth donor framework sequence having a high degree of homology with the selected framework region; and constructing a hybrid antibody variable domain or fragment thereof comprising the CDRs of the initial antibody and the donor framework sequences.

In certain embodiments, the method may additionally comprise, in no particular order, comparing the fourth donor framework sequence to germline sequences of said target species to identify a fourth germline framework sequence having a high degree of homology with the fourth framework sequence, and replacing at least one amino acid residue within the fourth donor framework sequence with the amino acid residue at the corresponding position in the fourth germline framework sequence.

In certain embodiments, the methods described herein may further comprise testing a hybrid antibody or antigen binding fragment thereof comprising any of the hybrid antibody variable domain or fragment thereof as described herein, to determine immunogenicity, or binding affinity, or both, relative to a hybrid antibody or antigen binding fragment thereof comprising a hybrid antibody variable domain or fragment thereof wherein the at least one amino acid within the first donor framework sequence has not been replaced with the corresponding amino acid residue from the first germline framework sequence.

In one embodiment, the hybrid antibody or antigen binding fragment thereof has a relative binding affinity of at least 60% of the initial antibody's affinity for said target.

In another embodiment, upon exposure to the immune system of the target species, the hybrid antibody or antigen binding fragment thereof has a reduced immunogenicity relative to a hybrid antibody or antigen binding fragment thereof wherein the at least one amino acid within the first donor framework sequence has not been replaced with the corresponding amino acid residue from the first germline framework sequence.

In certain embodiments, the hybrid antibody variable domain or fragment thereof is a variable domain of an antibody fragment selected from the group consisting of scFv, Fab, Fab′, F(ab′)₂, Fd, diabodies, antibody light chains and antibody heavy chains. In various embodiments, the target species may be human.

In a further aspect, a method for producing a hybrid antibody variable domain or fragment thereof is provided, wherein the method comprises (i) selecting a framework region from the group consisting of FR1, FR2, and FR3 within a variable region of an initial humanized antibody having specificity for a target; (ii) comparing the selected framework region sequence to human germline sequences to identify a first germline framework sequence having a high degree of homology with the first framework sequence; and (iii) modifying the selected framework region at one or more positions to introduce a mutation that changes an amino acid residue of selected framework region to the amino acid residue at the corresponding position of the germline framework sequence.

In certain embodiments, the method further comprises (i) selecting a second framework region from the group consisting of FR1, FR2, and FR3 of the initial humanized antibody; (ii) comparing the selected framework region sequence to human germline sequences to identify a second germline framework sequence having a high degree of homology with the second framework sequence; and (iii) modifying the selected framework region at one or more positions to introduce a mutation that changes an amino acid residue of the selected framework region to the amino acid residue at the corresponding position of the germline framework sequence.

In certain embodiments, said first and second framework sequences belong to the same germline gene family.

In another embodiment, the method additionally comprises (i) selecting a third framework region from the group consisting of FR1, FR2, and FR3 of the initial humanized antibody; (ii) comparing the selected framework region sequence to human germline sequences to identify a third germline framework sequence having a high degree of homology with the third framework sequence; and (iii) modifying the selected framework region at one or more positions to introduce a mutation that changes an amino acid residue of the selected framework region to the amino acid residue at the corresponding position of the germline framework sequence.

In certain embodiments, said third framework sequence belongs to the same germline gene family as the first framework sequence.

In yet another embodiment, the method further comprises (i) selecting a fourth framework region which is FR4 within a variable region of the initial humanized antibody; (ii) comparing the selected framework region sequence to human germline sequences to identify a fourth germline framework sequence having a high degree of homology with the fourth framework sequence; and (iii) modifying the selected framework region at one or more positions to introduce a mutation that changes an amino acid residue of the selected framework region to the amino acid residue at the corresponding position of the germline framework sequence.

In certain embodiments, the methods may also include testing a hybrid antibody or antigen binding fragment thereof comprising the hybrid antibody variable domain or fragment thereof as described herein to determine immunogenicity, or binding affinity, or both, relative to the initial humanized antibody.

In certain embodiments, the hybrid antibody or antigen binding fragment thereof has a relative binding affinity of at least 60% of the initial antibody's affinity for said target.

In certain embodiments, upon exposure to the immune system of the target species, the hybrid antibody or antigen binding fragment thereof has a reduced immunogenicity relative to the initial humanized antibody.

In another aspect, the disclosure provides a hybrid antibody or antigen binding fragment thereof specific for a target, which comprises (i) complementarity determining regions (CDRs) of an initial antibody, wherein said initial antibody is specific for said target, (ii) a first heavy chain framework region from a first antibody, and (iii) a second heavy chain framework region from a second antibody, wherein (a) the first and second antibodies belong to the same germline gene family, (b) the first and second heavy chain framework regions are selected from the group consisting of FR1, FR2, and FR3, (c) at least one of the heavy chain framework regions comprises a somatic hypermutation, (d) at least one of said first or second heavy chain framework regions comprises at least one mutation to an amino acid residue at the corresponding position of a germline framework sequence, and (e) the hybrid antibody or antigen binding fragment thereof is specific for said target.

In certain embodiments, said hybrid antibody or antigen binding fragment thereof further comprises a third heavy chain framework region selected from the group consisting of FR1, FR2, and FR3, wherein the third heavy chain framework region is from an antibody selected from the group consisting of the first antibody, the second antibody, and a third antibody which is neither the first nor the second antibody.

In certain embodiments, the third heavy chain framework region belongs to the same germline gene family as the first heavy chain framework region.

In certain embodiments, the hybrid antibody or antigen binding fragment thereof additionally comprises an FR4 heavy chain framework region from an antibody selected from the group consisting of the first antibody, the second antibody, the third antibody, and a fourth antibody which is neither the first, the second, nor the third antibody.

In certain embodiments, either, or both, of the third heavy chain framework region and the fourth heavy chain framework region belong to the same germline gene family as the first heavy chain framework region.

In certain embodiments, framework regions of the hybrid antibody or antigen binding fragment thereof are of human origin and the CDRs are of nonhuman origin.

In another aspect, the disclosure provides a hybrid antibody or antigen binding fragment thereof specific for a target, which comprises (i) complementarity determining regions (CDRs) of an initial antibody, wherein said initial antibody is specific for said target, (ii) a first light chain framework region from a first antibody, and (iii) a second light chain framework region from a second antibody, wherein (a) the first and second antibodies belong to the same germline gene family, (b) the first and second light chain framework regions are selected from the group consisting of FR1, FR2, and FR3, (c) at least one of the light chain framework regions comprises a somatic hypermutation, (d) at least one of said first or second light chain framework regions comprises at least one mutation to an amino acid residue at the corresponding position of a germline framework sequence, and (e) the hybrid antibody or antigen binding fragment thereof is specific for said target.

In certain embodiments, said hybrid antibody or antigen binding fragment thereof further comprises a third light chain framework region selected from the group consisting of FR1, FR2, and FR3, wherein the third light chain framework region is from an antibody selected from the group consisting of the first antibody, the second antibody, and a third antibody which is neither the first nor the second antibody.

In certain embodiments, the third light chain framework region belongs to the same germline gene family as the first light chain framework region.

In certain embodiments, the hybrid antibody or antigen binding fragment thereof additionally comprises an FR4 light chain framework region from an antibody selected from the group consisting of the first antibody, the second antibody, the third antibody, and a fourth antibody which is neither the first, the second, nor the third antibody.

In certain embodiments, either, or both, of the third light chain framework region and the fourth light chain framework region belong to the same germline gene family as the first light chain framework region.

In certain embodiments, framework regions of the hybrid antibody or antigen binding fragment thereof are of human origin and the CDRs are of nonhuman origin.

In certain embodiments, said light chain frameworks of the hybrid antibody or antigen binding fragment thereof are from a VL light chain. In other embodiments, said light chain frameworks of the hybrid antibody or antigen binding fragment thereof are from a VK light chain.

In certain embodiments, mutation of at least one amino acid residue of the hybrid antibody or antigen binding fragment to an amino acid residue at a corresponding position in a germline sequence as described herein confers reduced immunogenicity relative to a hybrid antibody that does not contain the mutation.

In certain embodiments, hybrid antibodies or antigen binding fragments thereof have a relative binding affinity of at least 60% of the initial antibody's affinity for a given target.

### BRIEF DESCRIPTION OF THE DRAWINGS

FIG. 1 is a chart depicting germ line genes of the Vκ gene locus. Vκ exon amino acid sequence alignment is shown. Alignments, numbering and loop regions are according to the structural criteria defined by Chothia. CDRs are according to Kabat, et al.

FIG. 2 is a chart depicting germline genes of the VH gene locus. VH exon amino acid sequence alignment is shown. Alignments, numbering and loop regions are according to the structural criteria defined by Chothia. CDRs are according to Kabat, et al.

FIG. 3 is a chart depicting germline genes of the Vλ gene locus. Vλ exon amino acid sequence alignment is shown. Alignments, numbering and loop regions are according to the structural criteria defined by Chothia. CDRs are according to Kabat, et al.

FIG. 4 is a chart depicting translated germline genes of the JH, JK and JL gene loci in terms of amino acid sequence alignment.

### DETAILED DESCRIPTION

### 1. Methods for Producing Hybrid Antibodies

In one aspect, the application provides methods for producing hybrid antibodies or antigen binding fragments thereof that involve altering the sequence of a donor framework region by replacing one or more amino acid residues in a donor framework region with an amino acid residue at a corresponding position in a framework germline sequence. Hybrid antibodies or antigen binding fragments thereof refer to antibodies or antigen binding fragments thereof that contain complementarity determining regions (CDRs) from an originating (initial) antibody of a first species in the functional context of framework regions selected from one or more antibodies of a target species. The hybrid antibodies or antigen binding fragments thereof retain at least a portion of the binding affinity for the target recognized by the initial antibody. The hybrid antibodies and antigen binding fragments thereof exhibit reduced immunogenicity when administered to a target species relative to a control antibody. An exemplary embodiment of a hybrid antibody is a humanized antibody.

When producing hybrid antibodies, the methods may utilize a known antibody to a given target as an initial or originating antibody. Alternatively, antibodies to a desired target may be generated using art recognized techniques. A variety of techniques for generating monoclonal antibodies directed to a desired target are well known to those skilled in the art. Examples of such techniques include, but are not limited to, those involving display libraries, xeno or huMAb mice, hybridomas, etc. Targets include any substance which is capable of exhibiting antigenicity and are usually proteins, carbohydrates, or glycosylated proteins. Examples of targets include receptors, enzymes, hormones, growth factors, peptides and the like. It should be understood that not only are naturally occurring antibodies suitable for use in accordance with the present disclosure, but engineered antibodies and antibody fragments which are directed to a predetermined target are also suitable as initial antibodies.

Antibodies (Abs) and antigen binding fragments useful as initial antibodies in accordance with the techniques set forth herein include monoclonal and polyclonal Abs, and antibody fragments such as Fab, Fab′, F(ab′)₂, Fd, scFv, diabodies, antibody light chains, antibody heavy chains and/or antibody fragments derived from phage or phagemid display technologies, as well as any humanized hybrid antibody generated according to any known methods in the art.

The originating species is any species used to generate the originating antibody or library from which the originating antibody was obtained. Exemplary originating species include, for example, rat, mouse, rabbit, chicken, monkey, human, etc.

The nucleic acid or amino acid sequence of the variable portion of the light chain, heavy chain or both, of an originating species antibody having specificity for a target antigen may be obtained using standard techniques of molecular biology. After a desired antibody is obtained, the framework (FR) and CDR regions of the variable regions (VH and VL) may be determined using any known definition of CDRs (e.g., Kabat alone, Chothia alone, Kabat and Chothia combined, and any others known to those skilled in the art). Once the FR and CDR regions of the originating antibody have been determined, FRs from one or more antibodies from a target species may be selected for use in constructing a hybrid antibody or antigen binding fragment thereof comprising the CDRs of the originating antibody and framework regions from one or more antibodies from a target species.

Framework regions from a target species for use in construction of a hybrid antibody may be selected by aligning one or more framework regions from the initial antibody sequence with variable amino acid sequences or antibody gene sequences from the target species to evaluate homology and/or identity. Programs for searching for alignments are well known in the art, e.g., BLAST and the like. For example, if the target species is human, a source of such amino acid sequences or gene sequences (germline or rearranged antibody sequences) may be found in any suitable reference database such as Genbank, the NCBI protein databank, VBASE (a database of human antibody genes maintained by the Medical Research Council; MRC Centre for Protein Engineering), and the Kabat database of immunoglobulins or translated products thereof. If the alignments are done based on the nucleotide sequences, then the selected genes should be analyzed to determine which genes of that subset have the closest amino acid homology to the originating species antibody.

The degree of homology is a measure of the relationship between two polypeptide sequences. In general, homology means a comparison between the amino acids of two polypeptide chains, on a residue by residue basis, taking into account not only exact correspondences between pairs of residues, one from each of the sequences being compared (as for identity) but also, where there is not an exact correspondence, whether, on an evolutionary basis, one residue is a likely substitute for the other. This likelihood has an associated “score” from which the % or degree of homology of the two sequences can then be determined. Methods for comparing the identity or homology of two or more sequences are well known in the art.

Suitable target species include any species to which one might want to administer the hybrid antibody or antigen binding fragment thereof. An exemplary target species is human. However, the target species is not limited to human and may include for example, monkey or other species.

### Overview of Framework Alignment and Hybrid Construction

In one aspect, the application provides methods for producing a hybrid antibody or antigen binding fragment thereof that involves modifying an initial antibody that has already been humanized (i.e., pre-humanized) according to known methods. In the simplest example, this may be an initial humanized antibody containing CDRs from a first non-human antibody, and framework regions from a second human antibody. As described herein, a framework region (i.e., FR1, FR2, FR3, or FR4) from the initial antibody is selected and aligned against a reference database of human germline sequences to identify a germline sequence having a high degree of homology with the framework sequence. The germline sequence with a high degree of homology with the framework sequence can be used to identify those amino acid positions within the framework sequence that have undergone somatic hypermutation relative to the germline sequence. Once such positions have been identified, one or more of the amino acid residues that reflect a somatic hypermutation may be selectively replaced with the amino acid residue at the corresponding position in the germline framework sequence. The method may involve making at least one modification to introduce a germline amino acid residue in at least one, at least two, at least three, or all four of the framework regions from the initial antibody. In various embodiments, at least one, two, three, four, five, or more modifications to introduce a germline amino acid residue may be made in a variable region sequence of an initial humanized antibody in order to produce a hybrid antibody.

In another aspect, the application provides methods for producing a hybrid antibody or antigen binding fragment thereof which involve replacing one or more selected framework regions of an initial antibody with a framework region from a target species, wherein the framework region of the target species is modified by changing at least one amino acid residue of the framework region to the amino acid residue of a germline framework sequence at the corresponding position within the framework sequence.

In one embodiment, a method for producing a hybrid antibody or antigen binding fragment thereof involves selecting an individual framework region of an initial antibody (not pre-humanized) from an originating species, i.e., FR1, FR2, FR3 or FR4, and aligning the selected framework region against candidate variable amino acid sequences or gene sequences from the target species in a reference database to identify a donor framework sequence. The donor framework sequence is then compared to a germline framework sequence from the same target species. By aligning the donor framework sequence with the germline framework sequence, it will be apparent which amino acid positions within the donor framework sequence have undergone somatic hypermutation relative to the germline framework sequence. Once such positions are identified, one or more of the amino acid residues at the positions involved in the somatic hypermutations are replaced with the amino acid residue from the corresponding position in the germline framework sequence. The modification of the donor framework sequence may be done either before or after the assembly of the hybrid antibody variable domain or fragment thereof containing the complementarity determining regions (CDRs) of the initial antibody and the donor framework sequence.

In another embodiment, a method for producing a hybrid antibody or antigen binding fragment thereof involves selecting donor framework sequences from a target species, constructing a hybrid antibody variable domain or fragment thereof, and then further modifying the hybrid antibody variable domain or fragment thereof by changing at least one amino acid residue in a framework region with the amino acid residue from a corresponding position in a germline framework sequence. In this method, an individual framework region of an initial antibody (not pre-humanized) from an originating species, i.e., FR1, FR2, FR3 or FR4, is selected and aligned against candidate variable sequences (either amino acid sequences or gene sequences) from the target species in a reference database to identify a donor framework sequence. The identified donor framework sequence is then used to construct a hybrid antibody variable domain comprising the CDRs of the initial antibody and the donor framework sequence. The donor framework sequence is then compared to a germline sequence from the same target species. By aligning the identified donor framework sequence to the germline framework sequence, it will be apparent which amino acid positions within the donor framework sequence have undergone somatic hypermutation relative to the germline framework sequence. Once such positions are identified, one or more of the amino acid residues at positions of the somatic hypermutations are replaced with the amino acid residue at the corresponding position in the germline framework sequence.

Donor framework regions from a target species are selected based on various criteria including degree of homology with a framework region from an originating antibody, the effect of the framework region on antigen binding affinity of the hybrid antibody relative to the originating antibody, and the effect of the framework region on the immunogenicity of the hybrid antibody relative to a control antibody. Typically, suitable donor framework regions from a target species will exhibit a high degree of homology (e.g., either amino acid or gene sequence homology) with a framework region from an originating antibody. In certain embodiments, a high degree of homology is at least 80%, 85%, 90%, 95%, or 98% homology. Suitable framework regions may also be identified based on the degree of identity (e.g., either amino acid or gene sequence identity) between a framework region of the originating antibody and a framework region of a target species. In certain embodiments, a high degree of identity is at least 65%, 70%, 75%, 80%, 85%, 90%, 97% or 98% identity.

When comparing a donor framework sequence from a target species to a germline sequence, the appropriate germline sequence to be used in the comparison may be determined in at least two ways. For example, based on knowledge about the antibody from which the donor framework sequence is obtained, one of skill in the art can determine the germline sequence from which the antibody was derived. Alternatively, the donor framework sequence (either amino acid sequence or nucleic acid sequence) may be compared to a database of germline sequences from the target species to identify a germline sequence having a high degree of homology (e.g., at least 80%, 85%, 90%, 95%, or 98% homology) or identity (e.g., at least 65%, 70%, 75%, 80%, 85%, 90%, 95%, 97% or 98%) with the donor framework sequence. In an exemplary embodiment, the germline framework sequence having the highest degree of homology or identity with the donor framework sequence is used for the comparison.

As described above, the methods for producing a hybrid antibody or antigen binding fragment thereof may involve replacing at least one of the framework regions from the initial antibody with a framework region from a donor antibody from a target species. In certain embodiments, the methods may involve replacing at least two, three or all four of the framework regions from the initial antibody with framework regions from one or more donor antibodies. Two or more framework regions may be replaced either sequentially or simultaneously when carrying out the methods described herein. Furthermore, the donor framework regions may be obtained from one antibody or from different antibodies. For example, in one embodiment, all four donor framework sequences may be obtained from the same antibody. In another embodiment, all four donor framework sequences are obtained from different antibodies. Every variation between these two extremes is also contemplated herein.

In an exemplary embodiment, a hybrid antibody or antigen binding fragment thereof comprises at least two donor framework sequences that are obtained from different antibodies belonging to the same germline gene family or two different antibodies derived from the same germline gene family member. Methods for framework patching using donor antibodies from the same germline gene families are described further below.

When replacing two or more framework regions, the specific method used to replace each framework region may be selected independently. For example, if the first framework region is replaced using the first embodiment described above, the second framework region may be replaced using either the first or second embodiment described above. Similarly, if the first framework region is replaced using the second embodiment described above, the second framework region may be replaced using either the first or second embodiment described above.

Illustrative methods for producing a hybrid antibody or hybrid antibody variable domain, or antigen binding fragments thereof, are described below. The following notations are used for clarity solely in these examples: FR1, FR2, FR3, and FR4 indicate framework regions from an initial antibody; *FR1*, *FR2*, *FR3*, and *FR4* indicate donor framework regions selected from a database of sequences of a target species; and fr1, fr2, fr3, and fr4 indicate donor framework regions that contain one or more of amino acid residues at positions of somatic hypermutations that have been replaced with the amino acid residue at the corresponding position in a germline framework sequence.

In a first example, FR1 is selected from a variable domain FR1-CDR1-FR2-CDR2-FR3-CDR3-FR4 of an initial antibody for alignment against candidate variable amino acid sequences or gene sequences from the target species in a reference database to identify a desirable donor framework region having a high degree of homology to FR1. The selected donor framework sequence, *FR1*, is then compared to germline sequences to identify a germline sequence having a high degree of homology to *FR1*. The *FR1* sequence and germline framework sequence are then aligned and positions of somatic hypermutation are identified. The *FR1* is then modified by replacing at least one amino acid residue at a position of somatic hypermutation within *FR1* with the amino acid at the corresponding position in the germline framework sequence to form fr1. The hybrid antibody may then be constructed to produce a hybrid antibody variable region or fragment thereof comprising fr1-CDR1-FR2-CDR2-FR3-CDR3-FR4. Alternatively, a hybrid antibody variable region comprising *FR1*-CDR1-FR2-CDR2-FR3-CDR3-FR4 may be first generated. The hybrid antibody variable region may then be modified by selectively replacing one or more residues within *FR1* that have undergone somatic hypermutation with corresponding germline amino acids to produce fr1-CDR1-FR2-CDR2-FR3-CDR3-FR4.

Additional framework regions may be replaced either sequentially or simultaneously using the methods described herein. For example, two, three or all four framework regions may be replaced. In an exemplary embodiment, all four framework regions are individually replaced to produce a hybrid antibody variable region or fragment thereof comprising fr1-CDR1-fr2-CDR2-fr3-CDR3-fr4.

When using the methods described herein, it is not necessary to replace all of the framework regions of the initial antibody with donor framework sequences. Furthermore, it is not necessary to make selective replacements to germline amino acids within all framework regions of the hybrid antibody. For example, according to the present notation, while a hybrid of the form fr1-CDR1-fr2-CDR2-fr3-CDR3-fr4 may be a suitable hybrid product, a hybrid of the form fr1-CDR1-fr2-CDR2-*FR3*-CDR3-*FR4* may be equally desirable.

In a second example, FR1 is selected from a variable domain FR1-CDR1-FR2-CDR2-FR3-CDR3-FR4 of an initial antibody. FR1 (amino acid or nucleic acid sequence) is then aligned against candidate variable amino acid sequences or gene sequences from the target species in a reference database to identify a desirable donor framework region having a high degree of homology to FR1. A hybrid antibody variable domain comprising *FR1*-CDR1-FR2-CDR2-FR3-CDR3-FR4 is then generated. The *FR1* is then compared to germline sequences to identify a germline sequence having a high degree of homology to *FR1*. The *FR1* sequence and germline framework sequence are then aligned and positions of somatic hypermutation are identified. The *FR1* is then modified by replacing at least one amino acid residue at a position of somatic hypermutation within *FR1* with the amino acid at the corresponding position in the germline framework sequence to produce fr1-CDR1-FR2-CDR2-FR3-CDR3-FR4. Additional framework regions may also be replaced, either sequentially or simultaneously, using the methods described herein.

In a third example, the process of producing a hybrid antibody or antigen binding fragment thereof can begin with an initial antibody that has already been humanized (i.e., pre-humanized). Again, using the notations described above, in this case, the initial antibody would be denoted as *FR1*-CDR1-*FR2*-CDR2-*FR3*-CDR3-*FR4*. The process of alignment of one or more *FR* regions against a germline database, selection of homologous germline sequences, selective replacement to generate the desired “fr” region(s), and construction of the final hybrid antibody or fragment thereof are similar to the methods described above. It should be apparent that a “donor” framework region as it applies to a pre-humanized initial antibody refers to those framework regions that occur in the pre-humanized antibody. The final product may have selective replacement of amino acid residues to germline residues in one, two, three or all four framework regions.

In certain embodiments, the hybrid antibodies and antibody binding fragments thereof produced in accordance with the methods described herein maintain at least a portion of the binding affinity for the target as compared to a control antibody. Suitable hybrid antibodies or antigen binding fragments thereof may maintain at least 50%, 60%, 75%, 80%, 85%, or 90% of the binding affinity of a control antibody. A control antibody is an antibody that is used as the basis for evaluating the effects on binding affinity of the hybridization process used to produce the hybrid antibody. An exemplary control antibody is the initial antibody. However, other control antibodies may also be appropriate depending on the particular circumstance. For example, in certain embodiments, it may be desirable to determine the effect of a framework replacement to a germline amino acid residue. In such circumstances, one could evaluate the binding affinity of the resulting hybrid antibody (e.g., with the germline replacement) to an equivalent hybrid antibody lacking the germline replacement. One of skill in the art will be able to determine an appropriate control antibody based on the disclosure herein. In certain embodiments, the methods described herein may further comprise a step of evaluating the binding affinity of the hybrid antibody or antigen binding fragment thereof relative to a control antibody. Binding affinity, association rate constants and dissociation rate constants for antibodies or antigen binding fragments thereof can be determined according to well known methods. Exemplary methods are described by, for example, Rother et al. (U.S. Pat. Nos. 7,399,594 and 7,393,648) and Queen et al. (U.S. Pat. Nos. 5,693,762, 6,180,371, and 7,022,500).

The hybrid antibodies and antigen binding fragments thereof produced in accordance with the methods described herein have reduced immunogenicity. Whether a hybrid antibody or antigen binding fragment has reduced immunogenicity may be determined relative to an appropriate control antibody for a target species. Reduced immunogenicity means that the immunogenicity of the hybrid antibody or antigen binding fragment thereof is reduced by at least 5%, 10%, 15%, 20%, 25%, 30%, 40%, 50%, 60%, 70%, 75%, 80%, 90%, or more relative to a control antibody. Suitable control antibodies include the initial antibody or a hybrid antibody or antigen binding fragment thereof not containing one or more framework replacements to germline amino acid residues. In certain embodiments, the methods described herein may further comprise a step of evaluating the immunogenicity of a hybrid antibody or antigen binding fragment thereof relative to a control antibody. The immunogenicity of an antibody in a target species can be assessed using the methods known in the art, such as human anti-mouse antibody (HAMA) ELISA assays.

Selective replacement of one or more amino acid positions within the donor framework sequence may be determined by selection factors that, when considered collectively, optimize the physiological and structural characteristics of the hybrid antibody or fragments thereof. In certain embodiments, effects on binding affinity and immunogenicity of the hybrid antibody or antigen binding fragment thereof may be taken into account when evaluating whether a position within a donor framework should be replaced with an amino acid residue from the corresponding position of a germline framework region. The effects of substitution of a given amino acid residue within the donor framework region on affinity and immunogenicity may be predicted in silico or determined empirically. Computer models are commonly used to identify amino acid positions within framework regions which are likely to interact with the CDR or a specific antigen, or both (see e.g., Whitelegg, N. R. & A. R. Rees: WAM: an improved algorithm for modeling antibodies on the WEB. Protein Eng., 13, 819-24 (2000)). This information can be used to determine whether the framework substitution is likely to have an effect on binding affinity. Similarly, computer programs are available which can predict whether a particular sequence is likely to be recognized as a T-cell antigen (see e.g., WO 00/34317 and US 2007/0292416; incorporated by reference herein). Such information can be used to determine whether the framework substitution is likely to have an effect on immunogenicity. In certain embodiments, combinations of in silico and empirical techniques may be used to evaluate whether a particular amino acid residue within a framework region should be changed to a germline amino acid residue.

The methods described herein involve replacing at least one amino acid residue in a donor framework region with an amino acid residue at a corresponding position within a germline framework region. In certain embodiments, at least two, three, four, five or more amino acid residues in a given donor framework region may be replaced with germline residues at corresponding positions. In certain embodiments, all of the donor framework amino acid residues that differ from a germline framework sequence may be changed to germline amino acid residues at corresponding positions. In certain embodiments, mutations in donor framework regions to germline amino acid residues may be made in a single donor framework region, or may be made in two, three or all four framework regions in a given variable domain.

It should be understood that a hybrid may be constructed using any combination of the above steps in any order. It is also noted that the selection of a first framework region within the initial antibody for any of the methods described above need not begin with FR1; rather, the first framework region selected for alignment may be FR2, FR3, or FR4. Similarly, each successive selection and alignment of framework regions need not occur in order of the framework numbers. Furthermore, it will be apparent that more than a single framework region may be selected and aligned against a reference database of sequences to identify a donor framework sequence having a high degree of homology. For example, FR1 and parts or all of the adjacent CDR1 sequence can be used for alignment, or, further still, the selection can also include the FR2 region of the initial antibody.

Hybrid antibodies or antigen binding fragments thereof that may be produced according to the methods described herein include full-length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also contemplated.

### Germline Gene Family Consideration for Framework Patching

In exemplary embodiments, the methods for producing hybrid antibodies or antigen binding fragments thereof described herein may involve framework patching with different antibodies from the same germline gene family or with different antibodies derived from the same germline gene family member. Methods for framework patching with considerations as to germline gene families are described in U.S. Pat. Nos. 7,393,648 and 7,399,594, which are hereby incorporated by reference.

Framework patching with germline gene family considerations first involves selection of candidate donor framework sequences. In particular, after alignment of a selected individual framework region of an initial antibody from an originating species, i.e., FR1, FR2, FR3 or FR4, against candidate variable amino acid sequences or gene sequences from the target species in a reference database, a set of donor framework sequences is identified. The set of identified donor framework sequences may comprise the top 100 hits, top 75 hits, top 50 hits, top 25 hits, top 10 hits, or top 5 hits, as determined by the database used. Alternatively, the set of donor sequences may comprise donor framework sequences having at least 80%, 85%, 90%, 95%, 98%, or 100% homology with the selected sequence from the initial antibody. In yet other embodiments, the set of donor sequences may comprise donor framework sequences having at least 65%, 70%, 75%, 80%, 85%, 90%, 97% or 100% identity to the selected sequence from the initial antibody. Homology and identity may be determined using either nucleic acid or amino acid sequences. Several selection criteria may be used to identify a desirable donor framework from any given set for further manipulation according to the present methods.

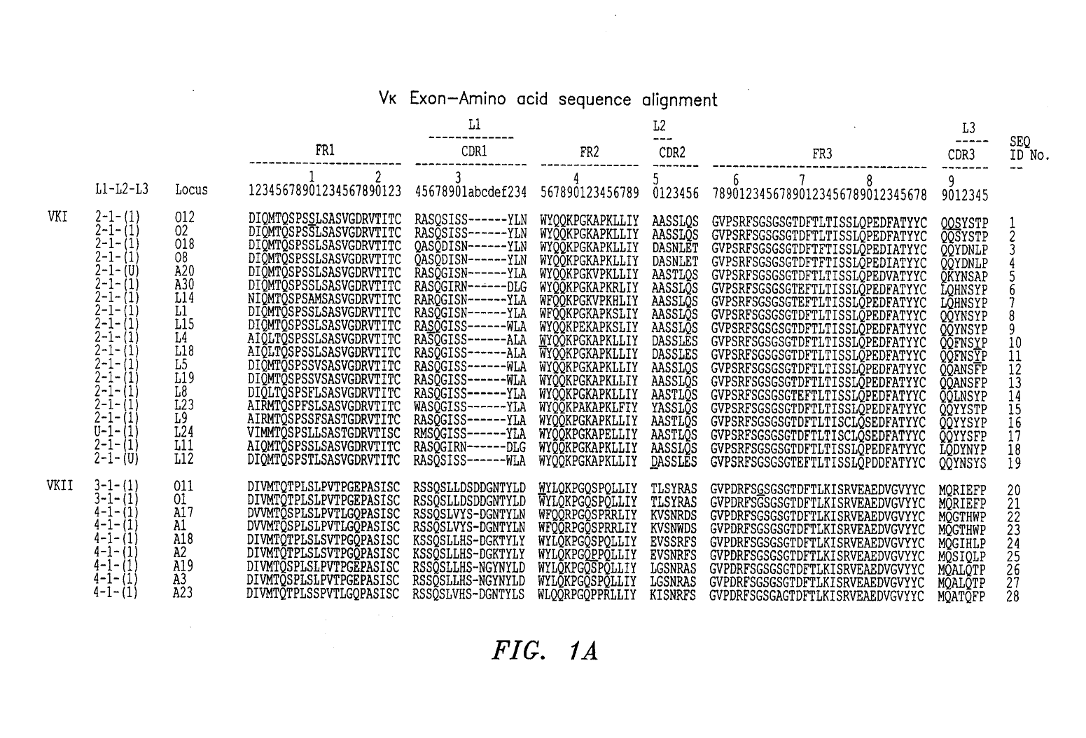

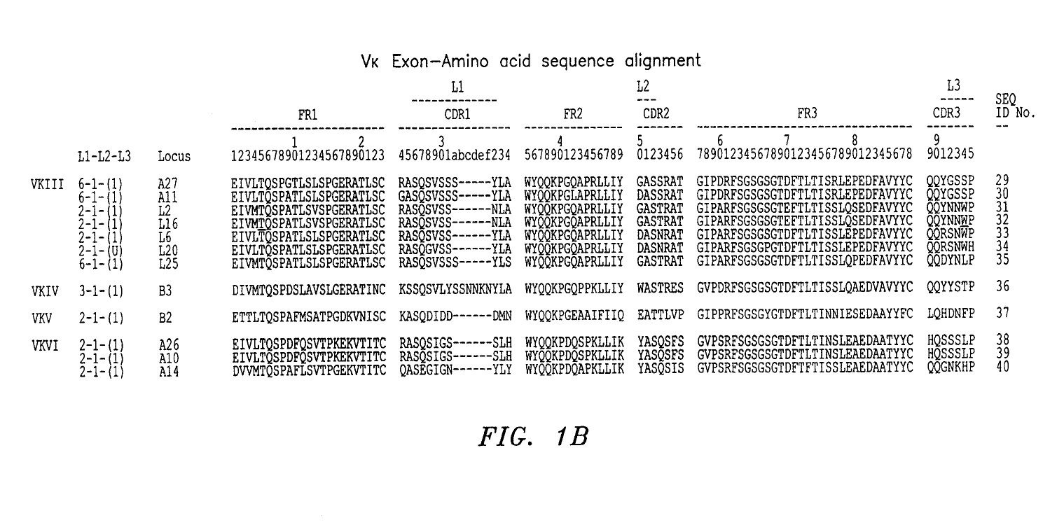

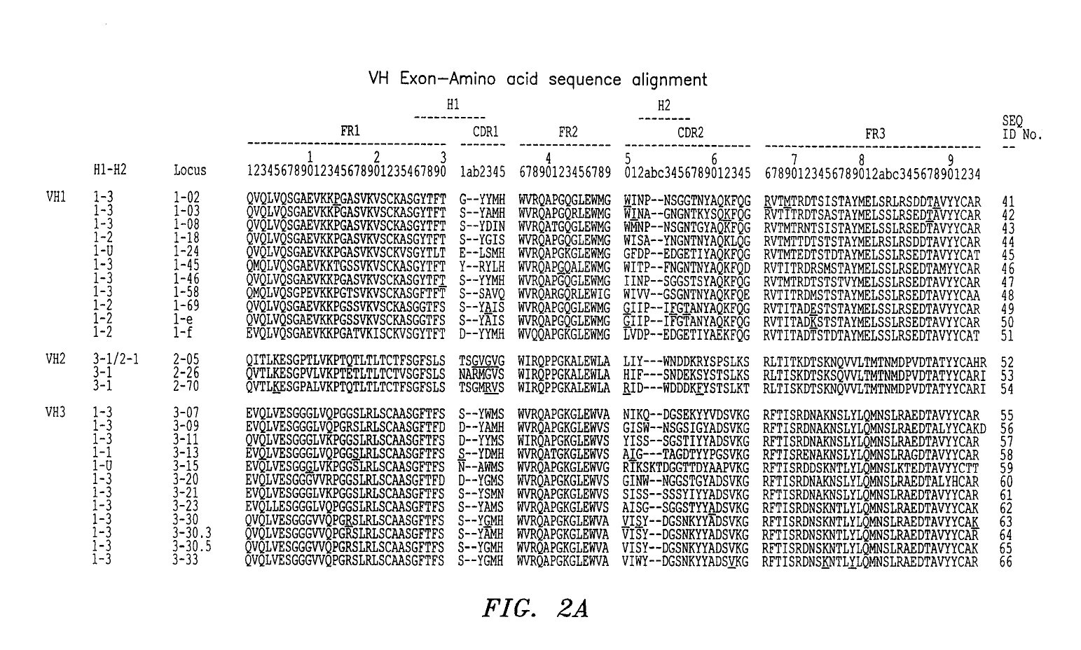

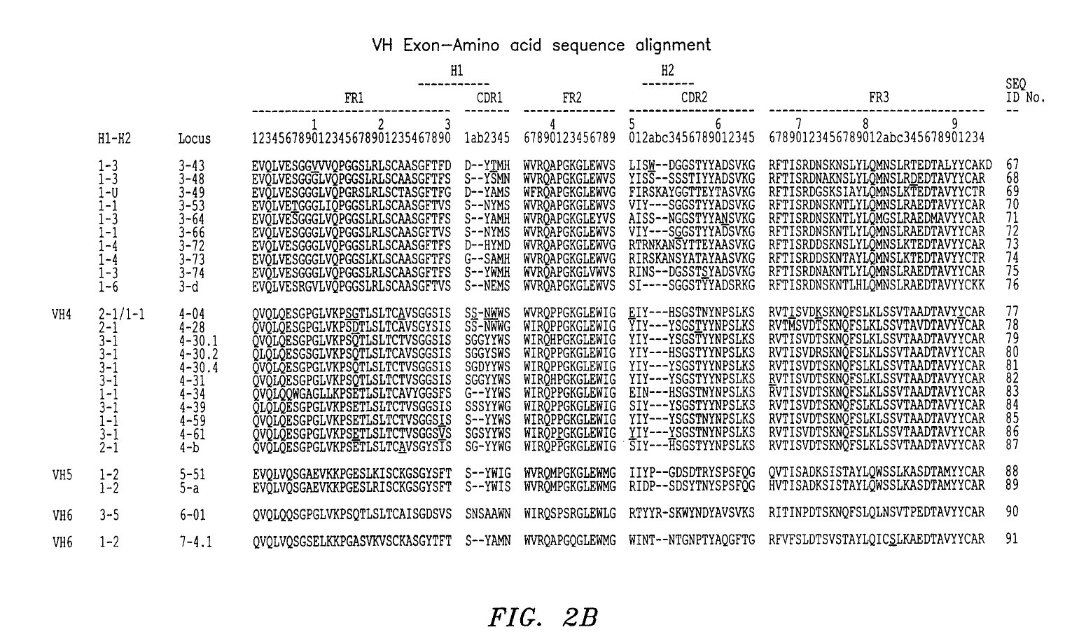

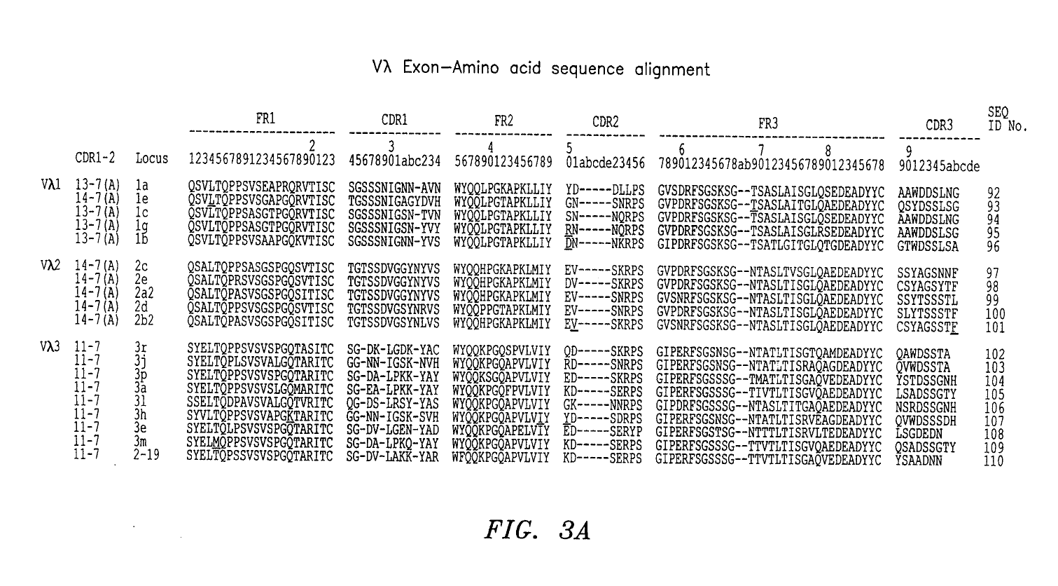

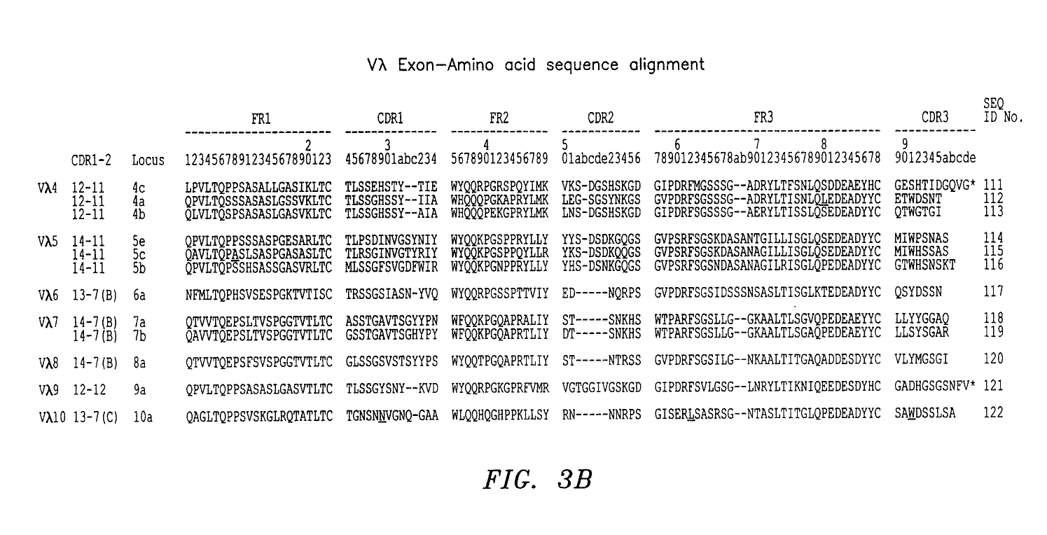

With respect to FR1, FR2, and FR3, the members of the set are categorized into original germline gene families, i.e., VH1, VH2, VH3, etc., VκI, VκII, VκIII, etc. and Vλ1, Vλ2, Vλ3, etc., and further, into germline gene family members where possible. See FIGS. 1, 2 and 3 for a more complete listing of germline gene families and germline gene family members. In certain embodiments, each donor framework sequence FR1, FR2, and FR3 may be derived from different germline gene families. However, although not always the case, the most similar sequence matches for each individual framework region may come from different antibodies or antibody fragments. In one embodiment, two or more framework regions come from different antibodies in the same germline gene family. In another embodiment, two or more framework regions come from different antibodies derived from (e.g., by somatic hypermutation) the same germline gene family member. In another embodiment, up to three framework regions can be from the same antibody. It is contemplated that even though there may be framework sequences in the database from a different germline gene family with a higher degree of homology, the more preferable candidate sequence may actually have lower homology but be from the same germline gene family as the other selected frameworks. Similarly, there may be framework sequences in the database from the same germline gene family with high homology, but from different germline gene family members of the same germline gene family. In certain embodiments, the more preferable donor framework candidates may be from the same family member as the other selected frameworks.

For example, if the FR1 donor framework sequence is from a heavy chain, the donor antibody from a target species may be characterized into one of the seven germline gene families for the heavy chain. By way of example, the framework region from the database may be characterized into the VH1 germline gene family which contains 11 germline gene family members. A second framework region from the initial antibody, for example FR2, is then compared to the database to identify an antibody containing a framework region suitable to serve as a donor framework sequence for FR2. The FR2 donor framework preferably originates from an antibody of a target species that is in the same germline gene family or even derived from the same germline gene family member as the antibody from which the FR1 donor framework sequence was derived. Thus, the antibody used as the source of donor framework sequence for FR2 is also characterized in the VH1 germline gene family as was the FR1 donor framework source.

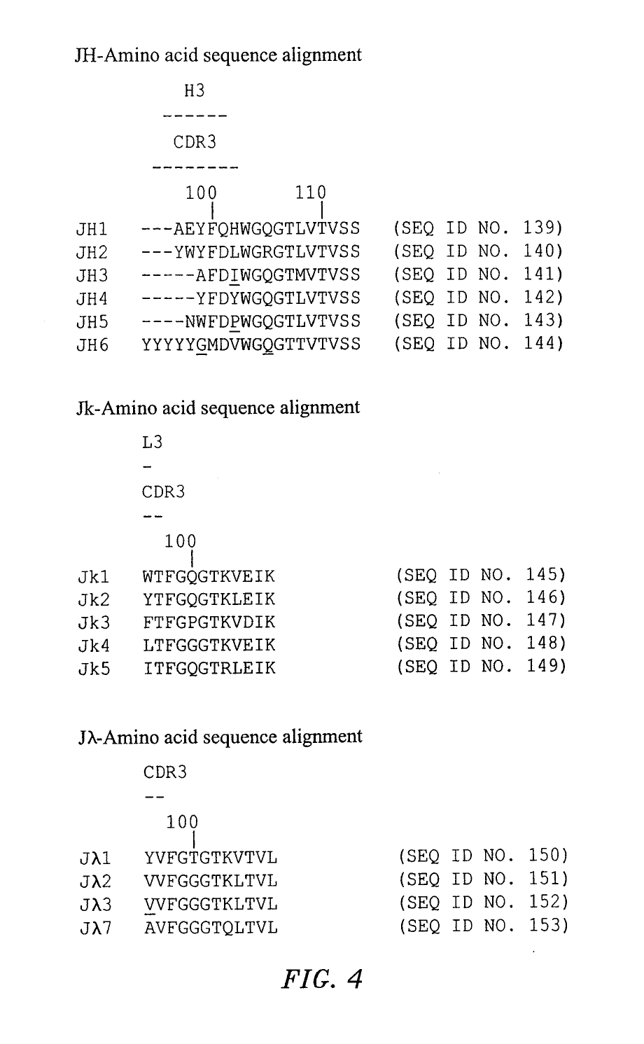

FR4 regions are not matched between families and family members of FR1, FR2, and FR3. Indeed, FR4 is encoded by J segments (See FIG. 4) and a choice of suitable FR4 sequences can be determined based on homology between the initial antibody FR4 sequences and the most similar FR4 donor sequences in a reference database. In one embodiment, the FR4 is chosen based on the degree of maximum homology between the initial antibody and those found in rearranged antibody sequence reference databases. In certain embodiments, 100% homology is preferred between the FR4 from the initial antibody and the FR4 selected from the reference database of the target species.

In certain embodiments of the invention, the selection of donor framework regions also includes an evaluation of amino acid positions in the CDRs as described in US 2003/0040606, which is incorporated herein by reference.

In certain embodiments, it is contemplated that at least two of the selected donor framework sequences may be obtained from different antibodies in the database. For example, FR1 may come from a first antibody, FR2 may come from a second antibody, FR3 may come from either the first or second antibody, or a third antibody which is neither the first or second antibody, and FR4 may come from the first, second, or third antibody, or a fourth antibody which is different from the first, second, and third antibodies, with the caveat that at least two FRs are from different antibodies. As another example, FR1 may come from a first antibody, FR3 may come from a second antibody, FR2 may come from either the first or second antibody, or a third antibody which is neither the first or second antibody, and FR4 may come from the first, second, or third antibody, or a fourth antibody which is different from the first, second, and third antibodies, with the caveat that at least two FRs are from different antibodies. As another example, FR1 may come from a first antibody, FR4 may come from a second antibody, FR2 may come from either the first or second antibody, or a third antibody which is neither the first or second antibody, and FR3 may come from the first, second, or third antibody, or a fourth antibody which is different from the first, second, and third antibodies, with the caveat that at least two FRs are from different antibodies. After selecting suitable framework region candidates, either or both the heavy and light chains variable regions are produced as further discussed below by grafting the CDRs from the originating species into the hybrid framework regions.

### Assembly of Hybrids

After selecting suitable donor framework region candidates according to the methods described herein, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) may be produced by oligonucleotide synthesis and/or PCR. The nucleic acid encoding CDR regions may also be isolated from the originating species antibodies using suitable restriction enzymes and ligated into the target species framework by ligating with suitable ligation enzymes.

Residues within donor framework regions can be replaced with germline amino acid residues according to standard methods of recombinant DNA technology. For example, a nucleic acid encoding a donor framework sequence having a modified sequence to account for a germline replacement may be made by oligonucleotide synthesis techniques. Alternatively, select positions within a nucleic acid encoding the donor framework region can be replaced (i.e. mutated) by site directed mutagenesis (e.g., PCR site-directed mutagenesis or cassette mutagenesis). For example, using cassette mutagenesis, a fragment within the donor framework region can be removed with one or more restriction enzymes of choice, and the corresponding desired germline fragment is ligated into the same site within the donor framework region. This ligation of the germline fragment sequence can result in the replacement of one or more positions, as desired, within the donor framework sequence that differ from the germline framework sequence. Alternatively, if it is desired to alter a donor framework region completely to a corresponding germline sequence, the germline sequence can be amplified by PCR and the resulting product can be used to assemble a hybrid antibody or antigen binding fragment thereof. It will be apparent that any combination of the methods known in the art may be used to generate a hybrid antibody. Such germline mutations may be made simultaneously with the construction of the hybrid variable region or may be after construction of a hybrid variable region. For example, in one embodiment, the donor framework regions may be first modified to introduce one or more changes to germline amino acid residues. The hybrid variable region may then be constructed using the CDRs from the initial antibody and the modified donor frameworks. Alternatively, a hybrid variable region comprising the CDRs from the initial antibody and the unmodified donor frameworks may first be constructed. The hybrid variable region is then modified to introduce one or more mutations to germline amino acid residues. Combinations of these methods are also envisioned, e.g., certain donor frameworks are modified prior to construction of the hybrid variable region and certain donor frameworks are modified after construction of the hybrid variable region.

Since the hybrids may be constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

Assembly of a physical antibody or antibody fragment library is preferably accomplished using synthetic oligonucleotides. In one example, oligonucleotides are designed to have overlapping regions so that they can anneal and be filled in by a polymerase, such as with polymerase chain reaction (PCR). Multiple steps of overlap extension are performed in order to generate the VL and VH gene inserts. Those fragments are designed with regions of overlap with human constant domains so that they can be fused by overlap extension to produce full length light chains and Fd heavy chain fragments. The light and heavy Fd chain regions may be linked together by overlap extension to create a single Fab library insert to be cloned into a display vector. Alternative methods for the assembly of the humanized library genes can also be used. For example, the library may be assembled from overlapping oligonucleotides using a Ligase Chain Reaction (LCR) approach. See, e.g., Chalmers and Curnow, Biotechniques (2001) 30-2, p249-252.

Various forms of antibody fragments may be generated and cloned into an appropriate vector to create a hybrid antibody library or hybrid antibody fragment library. For example variable genes can be cloned into a vector that contains, in-frame, the remaining portion of the necessary constant domain. Examples of additional fragments that can be cloned include whole light chains, the Fd portion of heavy chains, or fragments that contain both light chain and heavy chain Fd coding sequence. Alternatively, the antibody fragments used for humanization may be single chain antibodies (scFv).

Any selection display system may be used in conjunction with a library according to the present disclosure. Selection protocols for isolating desired members of large libraries are known in the art, as typified by phage display techniques. Such systems, in which diverse peptide sequences are displayed on the surface of filamentous bacteriophage (Scott and Smith (1990) Science, 249: 386), have proven useful for creating libraries of antibody fragments (and the nucleotide sequences that encode them) for the in vitro selection and amplification of specific antibody fragments that bind a target antigen. The nucleotide sequences encoding the VH and VL regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage or T7 capsids (phagebodies). An advantage of phage-based display systems is that, because they are biological systems, selected library members can be amplified simply by growing the phage containing the selected library member in bacterial cells. Furthermore, since the nucleotide sequence that encodes the polypeptide library member is contained on a phage or phagemid vector, sequencing, expression and subsequent genetic manipulation is relatively straightforward. Methods for the construction of bacteriophage antibody display libraries and lambda phage expression libraries are well known in the art (see, e.g., McCafferty et al. (1990) Nature, 348: 552; Kang et al. (1991) Proc. Natl. Acad. Sci. U.S.A., 88: 4363).

One display approach has been the use of scFv phage-libraries (see, e.g., Huston et al., 1988, Proc. Natl. Acad. Sci U.S.A., 85: 5879-5883; Chaudhary et al. (1990) Proc. Natl. Acad. Sci U.S.A., 87: 1066-1070. Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference. The display of Fab libraries is also known, for instance as described in WO92/01047 (CAT/MRC) and WO91/17271 (Affymax).

Hybrid antibodies or hybrid antibody fragments that are cloned into a display vector can be selected against the appropriate antigen in order to identify variants that maintain good binding activity because the antibody or antibody fragment will be present on the surface of the phage or phagemid particle. See for example Barbas III, et al. (2001) Phage Display, A Laboratory Manual, Cold Spring Harbor Laboratory Press, Cold Spring Harbor, N.Y., the contents of which are incorporated herein by reference. For example, in the case of Fab fragments, the light chain and heavy chain Fd products are under the control of a lac promoter, and each chain has a leader signal fused to it in order to be directed to the periplasmic space of the bacterial host. It is in this space that the antibody fragments will be able to properly assemble. The heavy chain fragments are expressed as a fusion with a phage coat protein domain which allows the assembled antibody fragment to be incorporated into the coat of a newly made phage or phagemid particle. Generation of new phagemid particles requires the addition of helper phage which contain all the necessary phage genes. Once a library of antibody fragments is presented on the phage or phagemid surface, a process termed panning follows. This is a method whereby i) the antibodies displayed on the surface of phage or phagemid particles are bound to the desired antigen, ii) non-binders are washed away, iii) bound particles are eluted from the antigen, and iv) eluted particles are exposed to fresh bacterial hosts in order to amplify the enriched pool for an additional round of selection. Typically three or four rounds of panning are performed prior to screening antibody clones for specific binding. In this way phage/phagemid particles allow the linkage of binding phenotype (antibody) with the genotype (DNA) making the use of antibody display technology very successful. However, other vector formats could be used for this humanization process, such as cloning the antibody fragment library into a lytic phage vector (modified T7 or Lambda Zap systems) for selection and/or screening.

After selection of desired hybrid antibodies and/or hybrid antibody fragments, it is contemplated that they can be produced in large volume by any technique known to those skilled in the art, e.g., prokaryotic or eukaryotic cell expression and the like. For example, hybrid antibodies or fragments may be produced by using conventional techniques to construct an expression vector that encodes an antibody heavy chain in which the CDRs and, if necessary, a minimal portion of the variable region framework, that are required to retain original species antibody binding specificity (as engineered according to the techniques described herein) are derived from the originating species antibody and the remainder of the antibody is derived from a target species immunoglobulin which may be manipulated as described herein, thereby producing a vector for the expression of a hybrid antibody heavy chain.

Additionally, an expression vector can be constructed that encodes an antibody light chain in which one or more CDRs and, if necessary, a minimal portion of the variable region framework, that are required to retain original species antibody binding specificity which may be manipulated as provided herein are derived from the originating species antibody, and the remainder of the antibody is derived from a target species immunoglobulin which can be manipulated as provided herein, thereby producing a vector for the expression of hybrid antibody light chain.

The expression vectors may then be transferred to a suitable host cell by conventional techniques to produce a transfected host cell for expression of optimized engineered antibodies or antibody fragments. The transfected or transformed host cell is then cultured using any suitable technique known to those skilled in the art to produce hybrid antibodies or hybrid antibody fragments.

In certain embodiments, host cells may be contransfected with two expression vectors, the first vector encoding a heavy chain derived polypeptide and the second encoding a light chain derived polypeptide. The two vectors may contain different selectable markers but, with the exception of the heavy and light chain coding sequences, are preferably identical. This procedure provides for equal expression of heavy and light chain polypeptides. Alternatively, a single vector may be used which encodes both heavy and light chain polypeptides. The coding sequences for the heavy and light chains may comprise cDNA or genomic DNA or both.

In certain embodiments, the host cell used to express hybrid antibodies or hybrid antibody fragments may be either a bacterial cell such as Escherichia coli, or preferably a eukaryotic cell. Preferably a mammalian cell such as a Chinese hamster ovary cell or NSO cell may be used. The choice of expression vector is dependent upon the choice of host cell, and may be selected so as to have the desired expression and regulatory characteristics in the selected host cell.

Once produced, the hybrid antibodies or hybrid antibody fragments may be purified by standard procedures of the art, including cross-flow filtration, ammonium sulphate precipitation, affinity column chromatography (e.g., protein A), gel electrophoresis and the like.

### 2. Hybrid Antibodies

In another aspect, the invention provides hybrid antibodies and antigen binding fragments thereof that contain at least one residue in a framework region that has been changed to an amino acid residue of a germline framework sequence at a corresponding position. Exemplary hybrid antibodies and antigen binding fragments thereof include those produced by the methods described herein.

In exemplary embodiments, the hybrid antibodies or antigen binding fragments thereof comprise both heavy and light chains wherein at least one amino acid residue of a framework region of the heavy chain and/or at least one amino acid residue of a framework region of the light chain have been changed to amino acid residues of a germline framework region at corresponding positions. In other embodiments, at least two, three, four, five or more residues of a given framework region have been changed to germline amino acid residues at corresponding positions. In certain embodiments, all amino acid residues in a given framework region that differ from a germline sequence can be changed to the corresponding germline amino acids. In any given hybrid antibody or antigen binding fragment thereof, changes to germline amino acid residues may be made in one, two, three or all four frameworks of the heavy chain and/or in one, two, three or all four framework regions of the light chain. In one embodiment, all four heavy chain framework regions and all four light chain framework regions comprise at least one mutation to a germline amino acid residue at a corresponding position.

In exemplary embodiments, the hybrid antibodies or antigen binding fragments thereof described herein are framework patched from at least two different antibodies from the same germline gene family or at least two different antibodies derived from the same germline gene family member.

In certain embodiments, the hybrid antibodies or antigen binding fragments described herein comprise at least one framework region having at least one residue that is a somatic mutation, e.g., a position that is not identical to an amino acid residue of a germline sequence at a corresponding position. For example, at least one framework region comprises an amino acid residue that is different from a germline amino acid residue at a corresponding position wherein the germline framework sequence and donor framework sequence are at least 70%, 80%, 85%, 90%, 95%, 97%, or 98% identical. Other hybrid antibodies or antigen binding fragments thereof comprise at least one somatic mutation in a framework region of a heavy chain and at least one somatic hypermutation in a framework region of a light chain. Still other hybrid antibodies or antigen binding fragments thereof comprise at least one somatic hypermutation in two, three, or all four framework regions of the light chain and/or at least one somatic hypermutation in two, three, or all four framework regions of the heavy chain. It should be understood that any given framework region may comprise at least one somatic hypermutation and at a different location at least one mutation such that an amino acid residue that originally differed from a germline sequence has been changed to the germline amino acid residue at the corresponding position.

In other embodiments, the hybrid antibody or antigen binding fragment thereof is specific for a target, and maintains at least a portion of the binding affinity of the initial antibody from which the CDRs were derived. The hybrid antibodies or antigen binding fragments thereof also exhibit reduced immunogenicity in comparison to a hybrid antibody or antigen binding fragment thereof that does not contain the changes to germline amino acids at corresponding positions in the framework regions.

In exemplary embodiments, the hybrid antibodies or antigen binding fragments thereof are humanized, e.g., they comprise donor framework regions from a human antibody and CDRs from a non-human species.

### 3. Hybrid Antibody Compositions and Methods of Use Thereof

The hybrid antibodies or hybrid antibody fragments may be used as therapeutic or diagnostic agents and may further be used in conjunction with or attached to other proteins (or parts thereof) such as human or humanized monoclonal antibodies. These other proteins may be reactive with other markers (epitopes) characteristic for a disease against which the antibodies are directed or may have different specificities chosen, for example, to recruit molecules or cells of the target species, e.g., receptors, target proteins, diseased cells, etc. The hybrid antibodies or antibody fragments may be administered with such proteins (or parts thereof) as separately administered compositions or as a single composition with the two agents linked by conventional chemical or by molecular biological methods. Additionally the diagnostic and therapeutic value of the antibodies may be augmented by labeling the antibodies with labels that produce a detectable signal (either in vitro or in vivo) or with a label having a therapeutic property. Some labels, e.g. radionuclides may produce a detectable signal and have a therapeutic property. Examples of radionuclide labels include ¹²⁵I, ¹³¹I, and ¹⁴C. Examples of other detectable labels include a fluorescent chromophore such as green fluorescent protein, fluorescein, phycobiliprotein or tetraethyl rhodamine for fluorescence microscopy, an enzyme which produces a fluorescent or colored product for detection by fluorescence, absorbance, visible color or agglutination, which produces an electron dense product for demonstration by electron microscopy; or an electron dense molecule such as ferritin, peroxidase or gold beads for direct or indirect electron microscopic visualization.

Hybrid antibodies or hybrid antibody fragments herein may typically be administered to a patient in a composition comprising a pharmaceutical carrier. A pharmaceutical carrier can be any compatible, non-toxic substance suitable for delivery of the monoclonal antibodies to the patient, sterile water, alcohol, fats, waxes, and inert solids may be included in the carrier. Pharmaceutically acceptable adjuvants (buffering agents, dispersing agent) may also be incorporated into the pharmaceutical composition.

The hybrid antibody or hybrid antibody fragment compositions may be administered to a patient in a variety of ways. Preferably, the pharmaceutical compositions may be administered parenterally, e.g., subcutaneously, intramuscularly or intravenously. Thus, compositions for parenteral administration may include a solution of the antibody, antibody fragment or a cocktail thereof dissolved in an acceptable carrier, preferably an aqueous carrier. A variety of aqueous carriers can be used, e.g., water, buffered water, 0.4% saline, 0.3% glycine and the like. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of antibody or antibody fragment in these formulations can vary widely, e.g., from less than about 0.5%, usually at or at least about 1% to as much as 15 or 20% by weight and will be selected primarily based on fluid volumes, viscosities, etc., in accordance with the particular mode of administration selected.

Actual methods for preparing parenterally administrable compositions and adjustments necessary for administration to subjects will be known or apparent to those skilled in the art and are described in more detail in, for example, Remington's Pharmaceutical Science, 17^{th} Ed., Mack Publishing Company, Easton, Pa. (1985), which is incorporated herein by reference.

Technical and scientific terms used herein have the meanings commonly understood by one of ordinary skill in the art to which the present teachings pertain, unless otherwise defined herein. Reference is made herein to various methodologies known to those of skill in the art. Publications and other materials setting forth such known methodologies to which reference is made are incorporated herein by reference in their entireties as though set forth in full. Practice of the methods described herein will employ, unless otherwise indicated, conventional techniques of chemistry, molecular biology, microbiology, recombinant DNA, and immunology, which are within the skill of the art. Such conventional techniques are explained fully in the literature. See, e.g., Sambrook, Fritsch, and Maniatis, Molecular Cloning; Laboratory Manual 2nd ed. (1989); DNA Cloning, Volumes I and II (D. N Glover ed. 1985); Oligonucleotide Synthesis (M. J. Gait ed, 1984); Nucleic Acid Hybridization (B. D. Hames & S. J. Higgins eds. 1984); the series, Methods in Enzymology (Academic Press, Inc.), particularly Vol. 154 and Vol. 155 (Wu and Grossman, eds.); PCR-A Practical Approach (McPherson, Quirke, and Taylor, eds., 1991); Immunology, 2d Edition, 1989, Roitt et al., C. V. Mosby Company, and New York; Advanced Immunology, 2d Edition, 1991, Male et al., Grower Medical Publishing, New York; DNA Cloning: A Practical Approach, Volumes I and II, 1985 (D. N. Glover ed.); Oligonucleotide Synthesis, 1984, (M. L. Gait ed); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; and Gene Transfer Vectors for Mammalian Cells, 1987 (J. H. Miller and M. P. Calos eds., Cold Spring Harbor Laboratory); WO97/08320; U.S. Pat. Nos. 5,427,908; 5,885,793; 5,969,108; 5,565,332; 5,837,500; 5,223,409; 5,403,484; 5,643,756; 5,723,287; 5,952,474; Knappik et al., 2000, J. Mol. Biol. 296:57-86; Barbas et al., 1991, Proc. Natl. Acad. Sci. USA 88:7978-7982; Schaffitzel et al. 1999, J. Immunol. Meth. 10:119-135; Kitamura, 1998, Int. J. Hematol., 67:351-359; Georgiou et al., 1997, Nat. Biotechnol. 15:29-34; Little, et al., 1995, J. Biotech. 41:187-195; Chauthaiwale et al., 1992, Microbiol. Rev., 56:577-591; Aruffo, 1991, Curr. Opin. Biotechnol. 2:735-741; McCafferty (Editor) et al., 1996, Antibody Engineering: A Practical Approach, the contents of which are incorporated herein by reference.

Any suitable materials and/or methods known to those of skill in the art can be utilized in carrying out the methods described herein; however, preferred materials and/or methods are described. Materials, reagents and the like to which reference is made in the description and examples are obtainable from commercial sources, unless otherwise noted.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended herein.

1. A method for producing a hybrid antibody variable domain or fragment thereof, comprising:
(i) selecting a framework region from the group consisting of FR1, FR2, and FR3 within a variable region of an initial antibody having specificity for a target;
(ii) comparing the selected framework region against candidate donor framework sequences from said target species to identify a first donor framework sequence having a high degree of homology with the selected framework region;
(iii) comparing the first donor framework sequence to germline sequences of said target species to identify a first germline framework sequence having a high degree of homology with the first donor framework sequence;
(iv) identifying at least one amino acid residue within the first donor framework sequence that is different from the amino acid residue at the corresponding position in the first germline framework sequence; and
(v) constructing a hybrid antibody variable domain or fragment thereof comprising the complementarity determining regions (CDRs) of the initial antibody and the first donor framework sequence, wherein the at least one amino acid residue within the first donor framework sequence is replaced with the amino acid residue at the corresponding position in the first germline framework sequence.

2. The method according to claim 1, further comprising:
selecting a second framework region from the group consisting of FR1, FR2, and FR3 of the initial antibody,
comparing the selected framework region against candidate donor framework sequences from said target species to identify a second donor framework sequence having a high degree of homology with the selected framework region; and
constructing a hybrid antibody variable domain or fragment thereof comprising the CDRs of the initial antibody and the donor framework sequences.

3. The method according to claim 2, further comprising:
comparing the second donor framework sequence to germline sequences of said target species to identify a second germline framework sequence having a high degree of homology with the second framework sequence; and
replacing at least one amino acid residue within the second donor framework sequence with the amino acid residue at the corresponding position in the second germline framework sequence.

4. The method according to claim 2, wherein said first and second donor sequences are from two different antibodies that belong to the same germline gene family.

5-21. (canceled)

22. The method according to claim 1 wherein the hybrid antibody variable domain or fragment thereof is a variable domain of an antibody fragment selected from the group consisting of scFv, Fab, Fab′, F(ab)₂, Fd, diabodies, antibody light chains and antibody heavy chains.

23. The method according to claim 1 wherein the target species is human.

24. A method for producing a hybrid antibody variable domain or fragment thereof, comprising:
(i) selecting a framework region from the group consisting of FR1, FR2, and FR3 within a variable region of an initial humanized antibody having specificity for a target;
(ii) comparing the selected framework region sequence to human germline sequences to identify a first germline framework sequence having a high degree of homology with the first framework sequence; and
(iii) modifying the selected framework region at one or more positions to introduce a mutation that changes an amino acid residue of selected framework region to the amino acid residue at the corresponding position of the germline framework sequence.

25. The method according to claim 24, further comprising:
(i) selecting a second framework region from the group consisting of FR1, FR2, and FR3 of the initial humanized antibody;
(ii) comparing the selected framework region sequence to human germline sequences to identify a second germline framework sequence having a high degree of homology with the second framework sequence; and
(iii) modifying the selected framework region at one or more positions to introduce a mutation that changes an amino acid residue of the selected framework region to the amino acid residue at the corresponding position of the germline framework sequence.

26. The method according to claim 24, wherein said first and second framework sequences belong to the same germline gene family.

27-32. (canceled)

33. A hybrid antibody or antigen binding fragment thereof specific for a target, comprising
(i) complementarity determining regions (CDRs) of an initial antibody, wherein said initial antibody is specific for said target,
(ii) a first heavy chain framework region from a first antibody, and
(iii) a second heavy chain framework region from a second antibody,
wherein the first and second antibodies belong to the same germline gene family,
wherein the first and second heavy chain framework regions are selected from the group consisting of FR1, FR2, and FR3,
wherein at least one of the heavy chain framework regions comprises a somatic hypermutation,
wherein at least one of said first or second heavy chain framework regions comprises at least one mutation to an amino acid residue at the corresponding position of a germline framework sequence, and
wherein the hybrid antibody or antigen binding fragment thereof is specific for said target.

34-37. (canceled)

38. The hybrid antibody or antigen binding fragment of claim 33, wherein the framework regions are of human origin and the CDRs are of nonhuman origin.

39. A hybrid antibody or antigen binding fragment thereof specific for a target, comprising
(i) complementarity determining regions (CDRs) of an initial antibody, wherein said initial antibody is specific for said target,
(ii) a first light chain framework region from a first antibody, and
(iii) a second light chain framework region from a second antibody,
wherein the first and second antibodies belong to the same germline gene family,
wherein the first and second light chain framework regions are selected from the group consisting of FR1, FR2, and FR3,
wherein at least one of the light chain framework regions comprises a somatic hypermutation,
wherein at least one of said first or second light chain framework regions comprises at least one mutation to an amino acid residue at the corresponding position of a germline framework sequence, and
wherein the hybrid antibody or antigen binding fragment thereof is specific for said target.

40-43. (canceled)

44. The hybrid antibody or antigen binding fragment of claim 33, wherein the framework regions are of human origin and the CDRs are of nonhuman origin.

45. The hybrid antibody or antigen binding fragment according to claim 39, wherein said light chain frameworks are from a VL light chain.

46. The hybrid antibody or antigen binding fragment according to claim 39, wherein said light chain frameworks are from a VK light chain.

47. The hybrid antibody or antigen binding fragment according to claim 33, wherein the mutation of at least one amino acid to the corresponding germline sequence confers reduced immunogenicity relative to a hybrid antibody that does not contain the mutation.

48. The hybrid antibody or antigen binding fragment according to claim 33, wherein said hybrid antibody or antigen binding fragment thereof has a relative binding affinity of at least 60% of the initial antibody's affinity for said target.
